(19) United States
(12) Patent Application Publication
(10) Pub. No.: US 2024/0269384 A1
(43) Pub. Date: Aug. 15, 2024

(54) SEPSIS MONITOR

(71) Applicant: MASIMO CORPORATION, Irvine, CA (US)

(72) Inventor: Massi Joe E. Kiani, Laguna Niguel, CA (US)

(21) Appl. No.: 18/641,190

(22) Filed: Apr. 19, 2024

Related U.S. Application Data
(63) Continuation of application No. 16/299,004, filed on Mar. 11, 2019.
Application No. 16/299,004 is a continuation of application No. 14/191,925, filed on Feb. 27, 2014, now Pat. No. 10,226,576.
Application No. 14/191,925 is a continuation of application No. 13/100,172, filed on May 3, 2011, now Pat. No. 8,663,107.
Application No. 13/100,172 is a continuation of application No. 11/803,936, filed on May 15, 2007, now Pat. No. 7,941,199.
(60) Provisional application No. 60/800,629, filed on May 15, 2006.

(51) Int. Cl. A61M 5/172; A61B 5/00; A61B 5/0205; A61B 5/022; A61B 5/0245; A61B 5/08; A61B 5/1455; A61B 7/04

(52) U.S. Cl. CPC A61M 5/1723; A61B 5/0205; A61B 5/022; A61B 5/02455; A61B 5/08; A61B 5/14551; A61B 5/412; A61B 5/6826; A61B 5/6838; A61B 7/04; A61B 5/082; A61B 2560/0276

(57) ABSTRACT

Sensors are attached to a living being so as to generate corresponding sensor signals. A monitor is in communications with the sensors so as to derive physiological parameters responsive to the sensor signals. Predetermined limits are applied to the physiological parameters. At least one indicator responsive to the physiological parameters and the predetermined limits signal the onset of a sepsis condition in the living being.

### CROSS-REFERENCE TO RELATED APPLICATIONS

The present application claims priority benefit under 35 U.S.C. § 120 to, and is a continuation of U.S. patent application Ser. No. 16/299,004, filed Mar. 11, 2019 entitled “Sepsis Monitor” (Attorney Docket No. MASCER.227C3) which is a continuation of U.S. patent application Ser. No. 14/191,925, filed Feb. 27, 2014 entitled “Sepsis Monitor” (Attorney Docket No. MASCER.227C2), which is a continuation of U.S. patent application Ser. No. 13/100,172, filed May 3, 2011 entitled “Sepsis Monitor”, now U.S. Pat. No. 8,663,107 (Attorney Docket No. MASCER.227C1), which is a continuation of U.S. patent application Ser. No. 11/803,936, filed May 15, 2007, entitled “Sepsis Monitor”, now U.S. Pat. No. 7,941,199 (Attorney Docket No. MASCER.227A), which claims priority benefit under 35 U.S.C. § 119(e) from U.S. Provisional Application No. 60/800,629, filed May 15, 2006, entitled “Septic Shock Monitor” (Attorney Docket No. MASCER.227PR). The present application also incorporates the foregoing disclosures herein by reference.

### BACKGROUND OF THE INVENTION

Sepsis is a serious medical condition caused by the body's response to an infection. The source of the infection can be any of a number of places throughout the body. Bacterial infections are the most common cause of sepsis, but sepsis can also be caused by fungal, parasitic, or viral infections. Toxins produced by an untreated or inadequately treated infection circulate in the bloodstream causing damage, for example, to the brain, heart, lungs, kidneys and liver. Severe sepsis can result in septic shock, a medical emergency in which the organs and tissues of the body are not receiving an adequate flow of blood.

### SUMMARY OF THE INVENTION

The signs and symptoms of sepsis may be subtle. The unacceptably low survival rate of severe sepsis indicates that current patient identification strategies may be lacking. For example, conventional patient monitors give insufficient advance warning of deteriorating patient health or the onset of potentially serious physiological conditions resulting from sepsis. Advantageously, a sepsis monitor noninvasively measures patient condition so as to provide caregivers with an advanced warning or prediction of the onset sepsis. A sepsis monitor may also be configured to provide automatic intervention or treatment of sepsis.

SIRS (systemic inflammatory response syndrome) refers to the systemic activation of the body's immune response, such as from sepsis. SIRS is manifested by, for example, the presence of more than one of a temperature greater than 38° C. or less than 36° C.; a heart rate greater than 90 beats/min.; and a respiration rate greater than 20 breaths/min. Thus, in an embodiment, a sepsis monitor is responsive to more than one of pulse rate, respiration rate and temperature.

Sepsis also results in large amounts of nitrous oxide (NO) released into the blood. It has been shown that NO functions, in part, as a killer molecule that is activated by immune cells. The overproduction of NO during sepsis induces excessive vascular relaxation and a profound hypotension that is also a characteristic feature of sepsis. NO interacts rapidly with hemoglobin to form methemoglobin (HbMet). Thus, HbMet can function as a marker for NO generation in patients with sepsis. Further, endogenously produced CO functions as a messenger molecule as part of a complex cascade of mediators resulting from sepsis. A portion of the endogenous CO is exhaled and a portion is present as carboxyhemoglobin (HbCO). Thus, in an embodiment, a sepsis monitor is responsive to one or more of HbCO, HbMet and blood pressure.

In an embodiment, sepsis monitoring is based upon one or more physiological parameters and associated parameter limits, trends, patterns and variability, alone or in combination. The physiological parameters may include: blood parameters derived from an optical sensor including one or more of oxygen saturation (Sp0₂), pulse rate, HbCO and HbMet; respiration rate (RR) derived from an acoustic sensor or a capnography sensor, as examples; noninvasive blood pressure (NIBP) derived from a blood pressure sensor, such as an inflatable cuff and corresponding acoustic sensor, a continuous NIBP (CNIBP) measurement device or an intelligent cuff inflation (ICI) device, to name a few; and temperature manually measured or derived from a thermistor or other temperature transducer.

One aspect of a sepsis monitor is sensors attached to a living being so as to generate corresponding sensor signals. A monitor is in communications with the sensors so as to derive physiological parameters responsive to the sensor signals. Predetermined limits are applied to the physiological parameters. At least one indicator responsive to the physiological parameters and the predetermined limits signal the onset of a sepsis condition in the living being.

Another aspect of a sepsis monitor is identifying physiological parameters indicative of an onset of a sepsis condition in a living being. Sensor signals are generated that are responsive to the physiological parameters. The physiological parameters are computed from the sensor signals. Predetermined rules are applied to the physiological parameters so as to determine the onset of the sepsis condition. An indicator signals the potential existence and likely nonexistence of the sepsis condition.

### BRIEF DESCRIPTION OF THE DRAWINGS

FIG. 1 is a general block diagram of a sepsis monitoring system;

FIGS. 2A-B are detailed diagrams of sepsis monitoring system embodiments;

FIG. 3 is an illustration of a sepsis monitoring system embodiment;

FIG. 4 is a general block diagram of a sepsis monitor incorporating a multiple parameter processor; and

FIG. 5 is a detailed block diagram of a multiple parameter processor embodiment.

### DETAILED DESCRIPTION OF THE PREFERRED EMBODIMENT

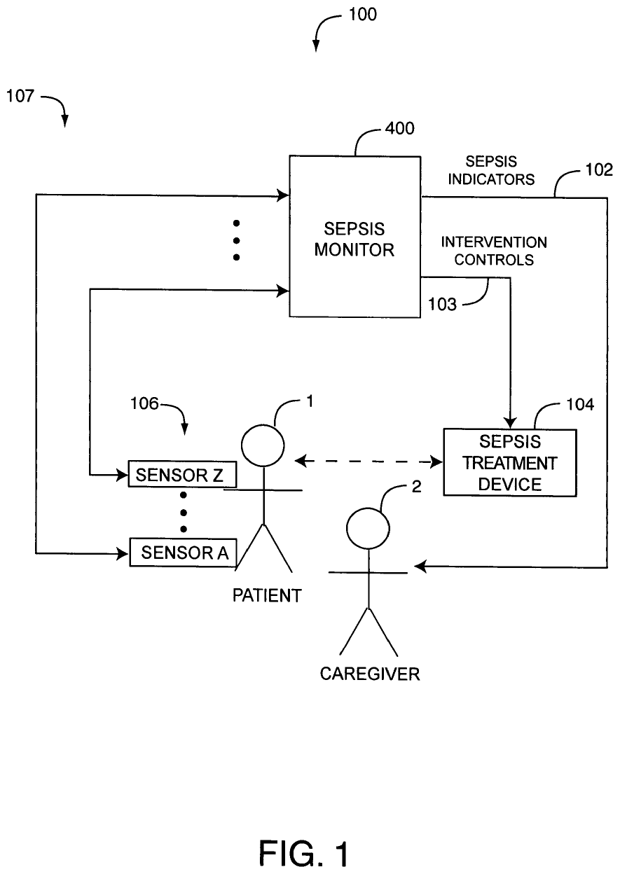

FIG. 1 illustrates a sepsis monitoring system 100 having one or more sensors 106 generating sensor signals 107 in response to physiological states of a living being, such as a patient 1. A sepsis monitor 400 processes the sensor signals 107 and generates sepsis indicators 102 or intervention controls 103 or both, in response. In an open-loop configuration, one or more sepsis indicators 102 are observed by a caregiver 2, who administers treatment in response. Alternatively, or in addition, the caregiver 2 initiates, pauses, halts or adjusts the settings of a sepsis treatment device 104 in response to the sepsis indicators 102. In an embodiment, the sepsis indicators 102 signal one or more of a prediction of the onset of sepsis, a sepsis condition, a prediction of the onset of septic shock and a septic shock condition. In a closed-loop configuration, the sepsis treatment device 104 is responsive to one or more intervention controls 103 so as to affect the treatment of the patient 1, including, for example, initiating, pausing, halting or adjusting the dosage of administered drugs. In an embodiment, the intervention controls 103 are responsive to one or more of a prediction of the onset of sepsis, a sepsis condition, a prediction of the onset of septic shock and a septic shock condition.

As shown in FIG. 1, the sepsis treatment device 104 may be a drug infusion device, a medical gas inhalation device or a ventilation device to name a few. Drug infusion device and gas inhalation device control is described in U.S. patent application Ser. No. 11/654,904, filed Jan. 17, 2007, entitled Drug Administration Controller and incorporated by reference herein. Closed loop respirator control is described in U.S. patent application Ser. No. 11/585,678, filed Oct. 23, 2006, entitled Robust Ventilator Control and incorporated by reference herein.

As shown in FIG. 1, sensors 106 provide noninvasive measurements and include, for example, an optical sensor attached to a tissue site, such as a fingertip, for measuring one or more blood parameters. Noninvasive sensors 106 may also include acoustic sensors, blood pressure cuffs, ECG or EEG electrodes, CO₂ measuring capnography sensors and temperature sensors to name but a few. The sepsis monitor 400 is responsive to sensors signals 107 so as to generate parameter measurements, which may include SpO₂, pulse rate, perfusion index, perfusion variability index, HbCO, HbMet, total hemoglobin, fractional saturation, glucose, cyanide, respiration rate, blood pressure, CO₂, bilirubin, lung volume, cardiac output, temperature, consciousness and hydration measures, among other parameters. Such parameters may be measured intermittently or continuously. Although sensors 106 are described above with respect to noninvasive technologies, sensors 106 may be invasive or noninvasive. Invasive measurements may require a person to prepare a blood or tissue sample, which is then processed by an instrument or testing device, with the result read from the instrument or device and manually entered into the sepsis monitor 400.

The sepsis monitor 400 may be a single instrument incorporating various hardware, software, circuits and code for processing sensor signals, deriving physiological parameters and processing those parameters to generate the indicators and controls described above. Alternatively, the sepsis monitor 400 may integrate one or more standalone instruments or plug-ins, each of which process specific sensor signals and derive particular physiological parameters. These may include blood parameter monitors, respiration rate monitors, blood pressure monitors, ECG and EEG monitors and capnometers, as a few examples.

In an embodiment, sensors 106 include a multiple wavelength optical sensor, such as described in U.S. patent application Ser. No. 11/376,013, filed Mar. 1, 2006 and entitled Multiple Wavelength Sensor Emitters; and the sepsis monitor 400 incorporates a patient monitor, such as described in U.S. patent application Ser. No. 11/367,033, filed Mar. 1, 2006 and entitled Noninvasive Multi-Parameter Patient Monitor, both patent applications assigned to Masimo Laboratories, Irvine, CA and both incorporated by reference herein.

In an embodiment, sensors 106 and measurement devices 108 include multiple wavelength sensors and corresponding noninvasive blood parameter monitors, such as Rainbow™ adhesive and reusable sensors and RAD-57™ and Radical-7™ monitors for measuring SpO₂, pulse rate, perfusion index, signal quality, HbCO and HbMet among other parameters. The Rainbow™ sensors and RAD-57™ and Radical-7™ monitors are available from Masimo Corporation, Irvine, CA. In an embodiment, sensors 106 include a pulse oximetry sensor, such as described in U.S. Pat. No. 5,782,757 entitled Low Noise Optical Probes and the sepsis monitor 400 incorporates a pulse oximeter, such as described in U.S. Pat. No. 5,632,272 entitled Signal Processing Apparatus, both assigned to Masimo Corporation, Irvine, CA and both incorporated by reference herein. In other embodiments, sensors 106 also include any of LNOP® adhesive or reusable sensors, SofTouch™ sensors, Hi-Fi Trauma™ or Blue™ sensor all available from Masimo Corporation, Irvine, CA. Further, the sepsis monitor 400 may also include any of Radical®, SatShare™, Rad-9™, Rad-5™, Rad-5v™ or PPO+™ Masimo SET® pulse oximeters all available from Masimo Corporation, Irvine, CA.

In another embodiment, the sepsis monitor 400 and the sepsis treatment device 104 are incorporated within a single unit. For example, the sepsis monitor 400 and treatment device 104 may be incorporated within a single housing, or the devices may be separately housed but physically and proximately connected.

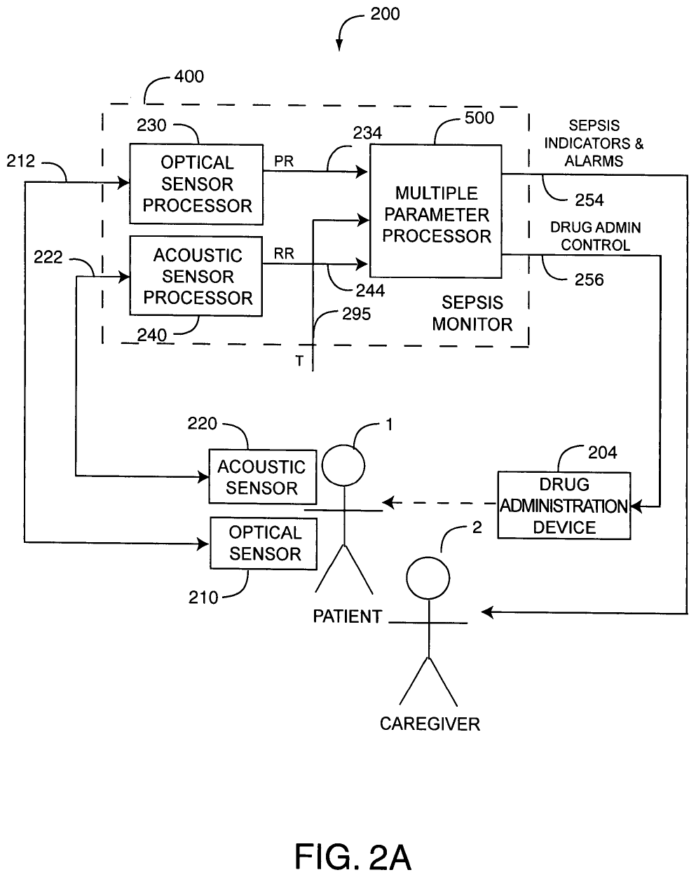

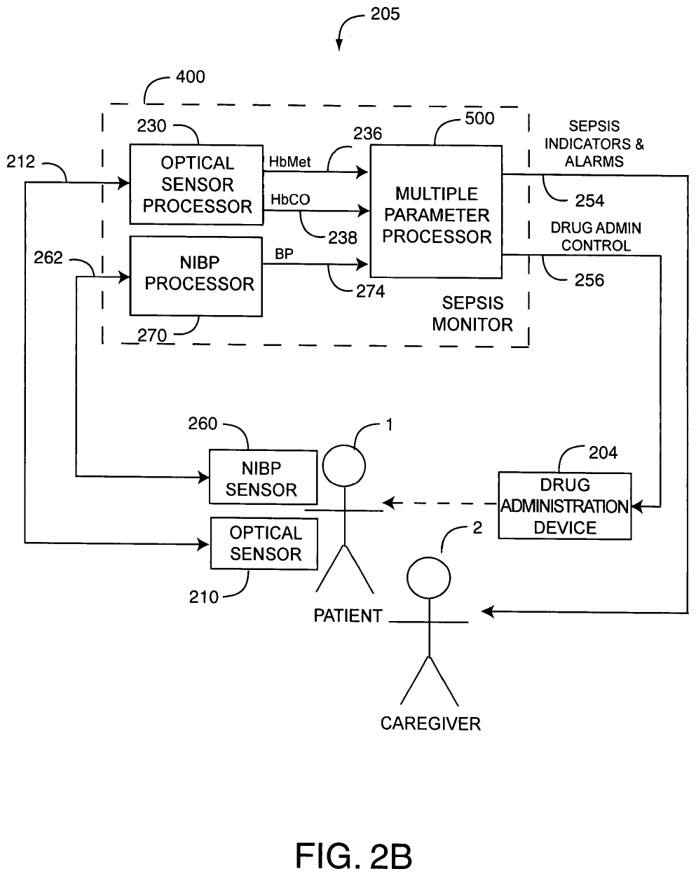

FIGS. 2A-B illustrate sepsis monitoring system embodiments 200, 205. As shown in FIG. 2A with respect to a system embodiment 200, a sepsis monitor 400 is in communications with an optical sensor 210 and an acoustic sensor 220 attached to a patient 1. An optical sensor processor 230 generates pulsatile-blood related parameters, such as pulse rate (PR) 234, in response to optical sensor signals 212. An acoustic sensor processor 240 generates body-sound related parameters 244, such as respiration rate (RR), in response to acoustic sensor signals 222. A temperature parameter 295 is generated via a temperature sensor or manually entered. A multiple parameter processor 500 processes the parameter measurements 234, 244, 295 alone or in combination and generates sepsis indicators and alarms 254 or drug administration controls 256, or both, in response. An acoustic sensor is described in U.S. Pat. No. 6,661,161 entitled Piezoelectric Biological Sound Monitor with Printed Circuit Board and a corresponding respirator rate monitor is described in International App. No. PCT/CA2005/000536 and PUb. No. WO 2005/096931, filed Apr. 8, 2005, both applications incorporated by reference herein.

As shown in FIG. 2B with respect to a system embodiment 205, a sepsis monitor 400 is in communications with an optical sensor 210 and a NIBP sensor 260 attached to a patient 1. An optical sensor processor 230 generates pulsatile-blood related parameters, such as such as HbCO 236 and HbMet 238 in response to optical sensor signals 212. An NIPB processor 270 generates blood pressure (BP) parameters, in response to NIBP sensor signals 262. A multiple parameter processor 500 processes the parameter measurements 236, 238, 274 alone or in combination and generates sepsis indicators and alarms 254 or drug adminstration controls 256, or both, in response. A continuous NIBP (CNIBP) sensor and processor are described in U.S. Pat. No. 5,590,649 entitled Apparatus and Method for Measuring an Induced Perturbation to Determine Blood Pressure and an intelligent cuff inflation (ICI) sensor and processor are described in U.S. Pat. No. 5,785,659 entitled Automatically Activated Blood Pressure Measurement Device, both patents incorporated by reference herein.

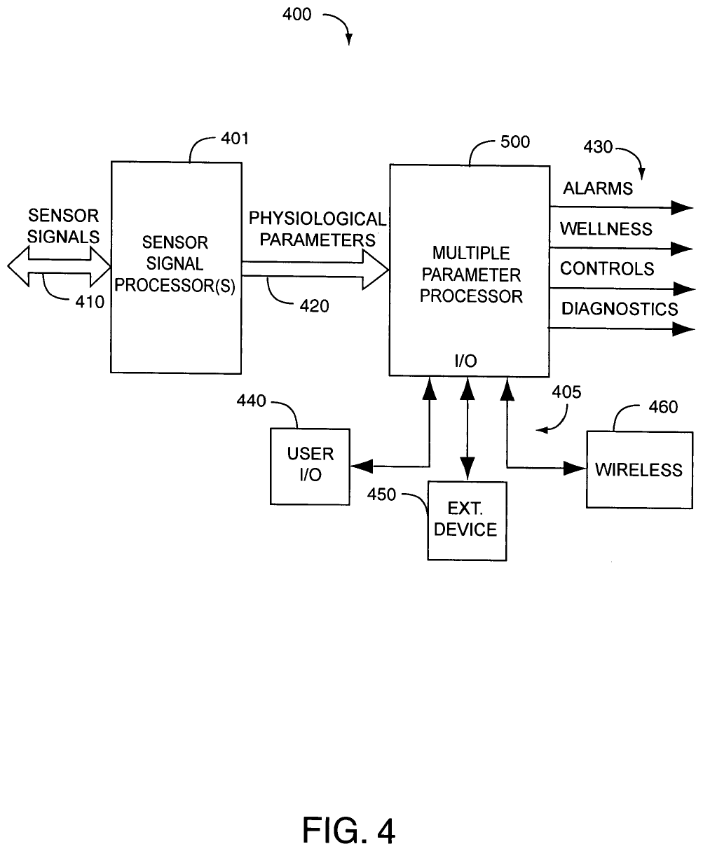

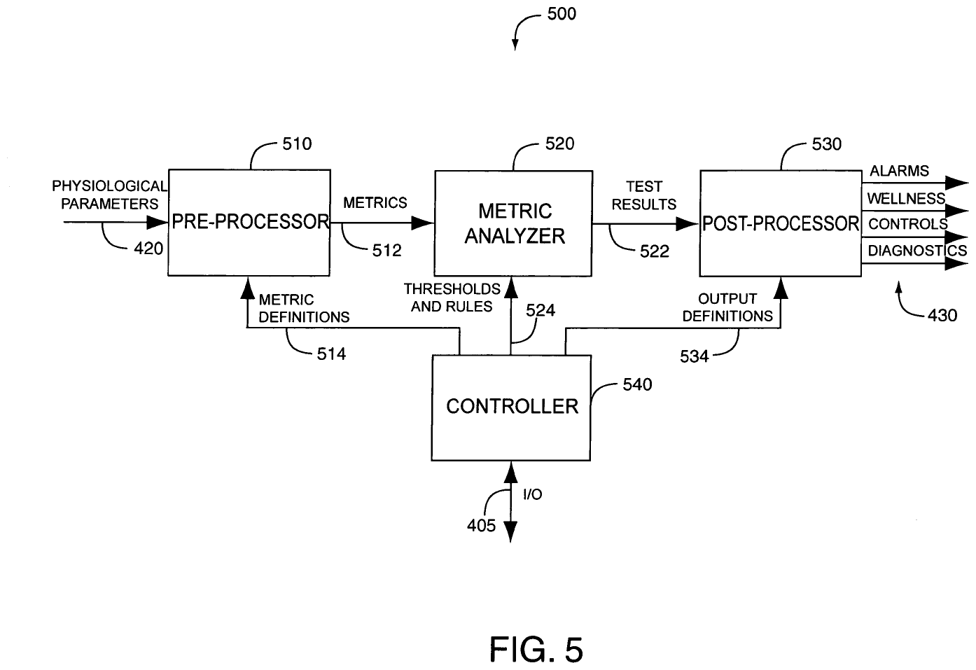

Advantageously, the multiple parameter processor 500 is responsive to a combination of multiple physiological parameters to indicate sepsis so that an alert can be provided based upon these parameters. Further, the multiple parameter processor 500 responds not only to parameter limits but also to parameter trend information, parameter patterns and parameter variability, so as to reflect a patient condition over time. In an embodiment, sepsis indicators 254 include alarms and wellness indicators that indicate stages of sepsis from none, to the onset of sepsis, to severe sepsis and septic shock. These outputs, for example, provide a warning of a potential onset of sepsis at an early stage and can trigger alarms as sepsis symptoms progress. Further, drug administration control 256 controls the administration of drugs or alters drug doses in response to patient condition. In an embodiment, the multiple parameter processor 500 compares parameter limits and rising or falling trends of the measurements 234, 244, 236, 238, 274, 295 alone or in combination, with corresponding predetermined thresholds and generates indicators and alarms 254 or drug administration controls 256 in response. The comparisons utilize a rule-based metric analysis, as described in detail in respect to FIGS. 4-5, below.

In one embodiment, the sepsis indicators 254 include a green indicator signaling a stable condition, a yellow indicator signaling a less stable condition or a potential sepsis onset and a red indicator signaling an unstable or severe sepsis condition. The indicators 254 may be, for example, various display LEDs emitting wavelengths of the appropriate colors. In an embodiment, a sepsis monitor 400 provides indicators 254 according to TABLES 1 and 2 below.

In an embodiment according to TABLE 1, below, if a patient's pulse rate (PR) and respiration rate (RR) are less than predetermined maximum limits and their body temperature is within a predetermined normal range, then the sepsis monitor 400 displays a green indicator. However, if more than one of pulse rate, respiration rate and body temperature are changing, where applicable changes in pulse rate and respiration rate are rate increases, then the sepsis monitor 400 displays a yellow indicator, signaling a potential onset of sepsis. If more than one of pulse rate, respiration rate and temperature become abnormal, including pulse rate and respiration rate above a predetermined limit and temperature outside of a predetermined range, then the sepsis monitor 400 displays a red indicator, signaling a potential sepsis condition.

TABLE 1
Rule-Based Monitor Outputs

| RULE | OUTPUT |
|---|---|
| If PR < heart rate limit; | Then illuminate green indicator. |
| RR < breathing rate limit; & | |
| T in normal range. | |
| If PR rising > heart rate trend limit; | Then illuminate yellow indicator |
| RR rising > breathing rate trend limit; | |
| & T rising or fallina. | |
| If PR > heart rate limit; | Then illuminate red indicator; |
| RR > breathing rate limit; & | Trigger audible alarm. |
| T outside normal range. | |

In an embodiment according to TABLE 2, below, if a patient's carboxyhemoglobin (HbCO), methemeglobin (HbMet) and blood pressure (BP) are normal, i.e. HbCO and HbMet less than predetermined maximum limits and BP greater than a predetermined minimum limit, then the sepsis monitor 400 displays a green indicator. However, if any of HbCO, HbMet and BP are changing, where applicable changes in HbCO and HbMet are increases and the applicable change in BP is a decrease, then the sepsis monitor 400 displays a yellow indicator, signaling a potential onset of sepsis. If any of HbCO, HbMet and BP change beyond predetermined limits, then the sepsis monitor 400 displays a red indicator, signaling a potential sepsis condition.

TABLE 2
Additional Rule-Based Monitor Outputs

| RULE | OUTPUT |
|---|---|
| If HbCO < CO limit; | Then illuminate green indicator. |
| HbMet < Met limit; & | |
| BP > blood pressure limit. | |
| If HbCO rising > CO trend limit; | Then illuminate yellow indicator |
| HbMet rising > Met trend limit or | |
| BP falling. | |
| If HbCO > HbCO limit threshold; | Then trigger an alarm, such as |
| HbMet & > HbMet limit threshold; | An audible or a visual alert or |
| or BP < blood pressure limit. | both. |

In other embodiments, a sepsis monitor 400 utilizes predetermined limits and ranges on any or all of PR, RR, T, HbCO, HbMet and BP to indicate no sepsis, a potential onset of sepsis or a sepsis condition, with green, yellow and red indicators or with other visual and audible indicators, displays and alarms. Other indicators, alarms, controls and diagnostics in response to various combinations of parameters and thresholds can be substituted for, or added to, the rule-based outputs illustrated in TABLES 1 and 2.

Other parameter measurements that may be input to the multiple parameter processor 500 include oxygen saturation (SpO₂) and perfusion index (PI) as derived from a pulse oximeter, EGG, EEG and ETC0₂, to name a few. All of these parameters may indicate real-time measurements and historical data such as measurement trends, patterns and variability. Signal quality measurements may also be input to the multiple parameter processor 500. Pulse oximetry signal quality and data confidence indicators are described in U.S. Pat. No. 6,684,090 entitled Pulse Oximetry Data Confidence Indicator, a pattern recognition alarm indicator is described in U.S. Pat. No. 6,822,564 entitled Parallel Measurement Alarm Processor, both patents assigned to Masimo Corporation, Irvine, GA and incorporated by reference herein.

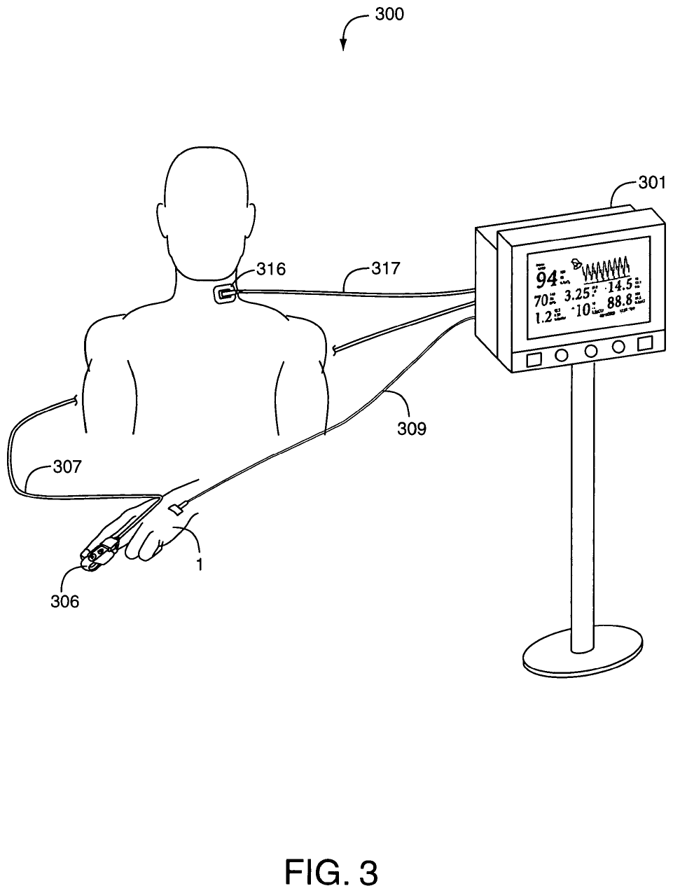

FIG. 3 illustrates a sepsis monitoring system 300 combining a sepsis monitor 400 (FIG. 1) and a drug administration device 204 (FIGS. 2A-B) into a drug infusion monitor 301. The sepsis monitoring system 300 has an optical sensor 306 and a piezoelectric sensor 316 attached to a patient's body 1. The optical sensor 306 detects pulsatile blood components and the piezoelectric sensor 316 detects tracheal sounds. The corresponding optical and acoustic sensor signals are transmitted to the drug infusion monitor 301 via an optical-sensor cable 307 and an acoustic-sensor cable 317. The drug infusion monitor 301 generates blood parameter measurements such as PR, HbCO and HbMet and biological sound measurements such as respiration rate (RR) and processes the measurements to display sepsis indicators and administer corresponding treatments. In a particular embodiment, the drug infusion monitor 301 intravenously administers one or more drugs, such as recombinant activated protein C, to the patient 1 in doses and dose intervals so as to respond to varying stages of sepsis or the potential onset of sepsis and to transitions between less severe and more severe stages of sepsis.

FIG. 4 illustrates a sepsis monitor embodiment 400 having sensor signal processor(s) 401, a multiple parameter processor 500, sensor signal inputs 410 to the signal processor(s) 401 and monitor outputs 430 from the parameter processor 500. Monitor outputs 430 may be sepsis alarms, wellness indicators, controls and sepsis diagnostics. Alarms may be used to alert medical personnel to a potential urgent or emergency medical condition in a patient under their care. Wellness indicators may be used to inform medical personnel as to patient condition stability or instability, such as a less urgent but potentially deteriorating medical state or condition. Diagnostics may be messages or other indicators used to assist medical personnel in diagnosing or treating a patient condition. Controls may be used to affect the operation of a medical treatment device, as described above, or other medical-related equipment.

In an embodiment, the multiple parameter processor 500 also has an input and output port (I/O) 405 that provides communications to the outside world. The I/O includes user I/O and external device communications to name a few. User I/O allows manual data entry and control. For example, a menu-driven operator display may be provided to allow entry of predetermined alarm thresholds. External device communications may include interfaces, networks or wireless communications to PCs, printers, chart recorders or displays to name a few.

FIG. 5 illustrates a sepsis monitor embodiment 500 having a pre-processor 510, a metric analyzer 520, a post-processor 530 and a controller 540. The pre-processor 510 has inputs 420 that may be real-time physiological parameter measurements, historical physiological parameter measurements, signal quality measures or any combination of the above. The pre-processor 510 generates metrics 512 that may include historical or real-time parameter trends, detected parameter patterns, parameter variability measures and signal quality indicators to name a few. As examples, trend metrics may indicate if a physiological parameter is increasing or decreasing at a certain rate over a certain time, pattern metrics may indicate if a parameter is cyclical within a particular frequency range or over a particular time period, variability metrics may indicate the extent of parameter stability.

As shown in FIG. 5, the metric analyzer 520 is configured to provide test results 522 to the post-processor based upon various rules applied to the metrics 512 in view of various thresholds 524. As an example, the metric analyzer 520 may output an alarm trigger 522 to the post-processor 530 when a parameter measurement 503 increases faster than a predetermined rate. This may be expressed, as an example, by a rule that states “if trend metric exceeds trend threshold then trigger alarm.” TABLE 1 and TABLE 2, above, illustrate sepsis monitor rules applied to metrics including PR, RR, T, HbCO, HbMet and BP parameters and trends.

Also shown in FIG. 5, the post processor 530 inputs test results 522 and generates outputs 502 including alarms, wellness indictors, controls and diagnostics. Alarms may be, for example, audible or visual alerts warning of critical conditions that need immediate attention. Wellness indicators may be audible or visual cues, such as an intermittent, low-volume tone or a red/yellow/green light indicating a patient with a stable or unstable physiological condition, as examples. Controls may be electrical or electronic, wired or wireless or mechanical outputs, to name a few, capable of interfacing with and affecting another device. Diagnostics may indicate a particular patient condition, such as the potential onset of sepsis.

Further shown in FIG. 5, the controller 540 interfaces with I/O 509. In one embodiment, the I/O 509 provides predetermined thresholds, which the controller 540 transmits to the metric analyzer 520. The controller 540 may also define metrics 514 for the pre-processor 510 and define outputs 534 for the post-processor 530.

A sepsis monitor has been disclosed in detail in connection with various embodiments. These embodiments are disclosed by way of examples only and are not to limit the scope of the claims that follow. One of ordinary skill in art will appreciate many variations and modifications.

1. (canceled)

2. (canceled)

3. (canceled)

4. (canceled)

5. (canceled)

6. (canceled)

7. (canceled)

8. (canceled)

9. (canceled)

10. A wellness monitor configured to alarm at an onset of a sepsis condition through monitoring a pulse rate, respiration rate and temperature of a wearer, the wellness monitor comprising:
one or more sensors configured to be applied to a wearer, the one or more sensors configured to output a plurality of signals responsive to a noninvasive measurements, one or more of the plurality of signals including data sufficient to determine a pulse rate, a respiration rate and a temperature of the wearer;
one or more processors configured to receive and process said data, electronically determine said pulse rate, said respiration rate, and said temperature, and output at least one of an audio or visual sepsis alarm responsive to said determination, said processing including comparing measurement values and comparing measurement trends,
wherein said comparing measurement values includes a first type of said sepsis alarm responsive to a combination only of (i) said pulse rate being above a pulse rate threshold, (ii) said respiration rate being above a respiration rate threshold, and (iii) said temperature being above a high temperature threshold or being below a low temperature threshold, and
wherein said comparing measurement trends includes a second type of said sepsis alarm responsive to a combination only of (a) a pulse rate trend of said pulse rate rising or being above said pulse rate threshold, (b) a respiration rate trend of said respiration rate rising or being above said respiration rate threshold, and (c) a temperature trend rising or being above said high temperature threshold or said temperature trend falling or being below said low temperature threshold, and
wherein said first type of said sepsis alarm is different from said second type of said sepsis alarm, said pulse rate threshold corresponds to an abnormal pulse rate, said respiration rate threshold corresponds to an abnormal respiration rate, and said high and low temperature thresholds each correspond to respective abnormal temperatures.

11. The monitor of claim 10, wherein said one or more sensors comprises a noninvasive pulse oximetry sensor.

12. The monitor of claim 10, wherein said one or more processors are further configured to output a drug control signal configured to cause a drug administration device to one of deliver medication to said wearer, adjust the delivery of medication to said wearer or cease the delivery of medication to said wearer.

13. The monitor of claim 10, wherein said one or more processors are further configured to output a drug administration instruction to a caregiver, said instruction configured to inform the caregiver about the administration of medication to said wearer.

14. An application configured to execute on a mobile computing device, the application configured to generate notifications, a first notification corresponding to a set of specific physiological parameter measurements all being abnormal, a second notification corresponding to a set of specific trends of physiological parameter measurements all being abnormal, the first and second notifications being different flags of a sepsis condition of a wearer of noninvasive sensors including at least a noninvasive pulse oximetry sensor, the application comprising:
a first electronic signal input responsive to a currently monitored pulse rate of the wearer;
a second electronic signal input responsive to a currently monitored respiration rate of the wearer;
a third electronic signal input responsive to a currently monitored temperature of the wearer;
a first electronic output configured to generate the first notification, the first notification corresponding to a combination only of (i) said pulse rate being above a pulse rate threshold corresponding to an abnormal pulse rate, (ii) said respiration rate being above a respiration rate threshold corresponding to an abnormal respiration rate, and (iii) said temperature being above a high temperature threshold or being below a low temperature threshold, the high and low temperature thresholds corresponding to abnormal temperatures; and
a second electronic output configured to generate the second notification, the second notification corresponding to a combination only of (a) a pulse rate trend of said pulse rate rising or being above said pulse rate threshold, (b) a respiration rate trend of said respiration rate rising or being above said respiration rate threshold, and (c) a temperature trend rising or being above said high temperature threshold or said temperature trend falling or being below said low temperature threshold.

15. The application of claim 14, wherein the first notification is configured to deliver medication to said wearer, adjust the delivery of medication to said wearer or cease the delivery of medication to said wearer.

16. The application of claim 14, wherein the second notification is configured to deliver medication to said wearer, adjust the delivery of medication to said wearer or cease the delivery of medication to said wearer.

17. The application of claim 14, wherein the first notification is configured to electronically deliver drug administration instructions to a caregiver.

18. The application of claim 14, wherein the second notification is configured to electronically deliver drug administration instructions to a caregiver.
